(19) United States
(12) Patent Application Publication
(10) Pub. No.: US 2011/0178592 A1
(43) Pub. Date: Jul. 21, 2011

(54) Implantable Tube And Coating Method Thereof

(75) Inventors: Dae-Joong Kim, Gyeonggi-do (KR); In-Su Baek, Gyeonggi-do (KR); Chul-Soo Gim, Gyeonggi-Do (KR); Sang-Ho Kim, Seongnam-Si (KR)

(73) Assignee: M.I.Tech Co., Inc., Pyeongtaek Si, Gyeonggi-Do (KR)

(21) Appl. No.: 12/922,674

(22) PCT Filed: Sep. 29, 2009
(86) PCT No.: PCT/KR2009/005578

§ 371 Date: Sep. 14, 2010

(51) Int. Cl. A61F 2/01; B05D 7/00

(52) U.S. Cl. 623/1.42; 427/2.25; 623/1.46

(57) ABSTRACT

An implantable tube to be implanted in a human body so as to be used as a circulation path, and a coating method thereof are disclosed. The implantable tube is a structure with a lumen coated with a bioactive material containing a material promoting fusing of the structure implanted in the body with ambient tissues. When the structure coated with a bioactive material is implanted in the body, the reaction of fusing the structure with ambient tissues can be promoted to shorten a stabilization period required until dialysis begins. In addition, since a chemical drug, medicament for inducing the generation of a growth factor promoting fusing of the structure with ambient tissues, is used, the structure can be easily coated.

### BACKGROUND OF THE INVENTION

1. Field of the Invention

The present invention relates to an implantable tube to be implanted in a human body so as to be used as a circulation path, and a coating method thereof.

2. Background of the Invention

In general, hemodialysis, a dialysis treatment used to a renal replacement therapy for patient, is substituting a major kidney function by removing waste products containing nitrogen from the blood of the patient and circulating part of the blood of the patient with a dialysis machine (i.e., an artificial kidney machine).

In order to perform hemodialysis, a passage for a dialysis treatment must be made in the blood. That is, a place, at which arterial blood is to be drawn out of the patient, connected to the artificial kidney, and the dialyzed blood is returned to the patient, must be installed at the vein side.

A blood accessing method includes an outer shunt method using a scrivener shunt in which a cannula made of plastic is inserted in an artery and vein, an inner shunt method in which body fluids are extracted by inputting a hollow, fine needle whenever dialysis is performed on a vein enlarged by anastomosing the artery and vein, an artificial blood vessel implantation method, and the like.

Meanwhile, an artificial blood vessel has been developed as a substitute for guiding the flow of a blood vessel of a patient when the blood vessel of the patient has been narrowed due to certain factors or the function of the blood vessel has been degraded, and a patency rate of the artificial blood vessel is affected by a chemical component, physical characteristics, elasticity, and the aspect of a surface structure of the artificial blood vessel.

However, when the artificial blood vessel implantation is employed to receive a hemodialysis treatment, the blood vessel is constricted at the portion connecting the artificial blood vessel and the artery and vein of the patient to interfere with the flow of the blood. To resolve this problem, the inventor of the present application filed a technique regarding a tube for connecting the artery and vein of a hemodialysis patient with medicament surface-treatment, which was registered as Korean Patent Registration No. 10-0596218.

The registered patent relates to a tube connecting the artery and vein of a hemodialysis patient, disclosing a technique of surface-processing the inner and outer surfaces of an artificial blood vessel with medicament in order to improve constriction at the portion connecting the artery and vein due to intimal hyperplasia.

Meanwhile, besides the problem of improving constriction, the use of the method of using implantation of an artificial blood vessel requires a two to four week stabilization period to implant an artificial blood vessel and perform the first hemodialysis. This is because, the scar at or around the anastomosis between the blood vessel of the patient and the artificial blood vessel must be healed and the myofibroblast needs to proliferate to a degree between the implanted artificial blood vessel and the ambient tissues, so that the artificial blood vessel can be fused with the ambient tissues and fixed.

### SUMMARY OF THE INVENTION

The present invention solves the above problems, and provides an implantable tube capable of promoting the fusing of an implanted structure with ambient tissues, and a coating method thereof.

In accordance with an exemplary embodiment of the present invention, there is provided an implantable tube as a structure with a lumen coated with a bioactive material, wherein the bioactive material contains a material promoting fusing of the structure implanted in the body with ambient tissues.

Preferably, the material for promoting fusing of the structure, implanted in the body, with ambient tissues is a material for promoting the creation of a blood vessel. The material for promoting fusing of the structure, implanted in the body, with ambient tissues is a material for promoting the proliferation of myofibroblast.

Preferably, the material for promoting proliferation of myofibroblast is one of Vascular Endothelial Growth Factor (VEGF), Epidermal Growth Factor (EGF), Platelet Derived Growth Factor (PDGF), and Fibroblast Growth Factor (FGF).

Preferably, the bioactive material is coated on an outer surface of the structure. The bioactive material may further include medicament suppressing neointimal hyperplasia.

Preferably, the medicament suppressing the neointimal hyperplasia is coated on an inner surface of the structure.

Preferably, the structure is a thin film of Expanded Polytetrafluoroethylene (e-PTFE) having fine pores.

Preferably, the material for promoting fusing of the structure, implanted in the body, with ambient tissues is generated by medicament that induces generation of a material promoting fusing with ambient tissues.

Preferably, the medicament that induces the generation of the material for promoting fusing of the structure, implanted in the body, with ambient tissues is a chemical drug.

In accordance with another exemplary embodiment of the present invention, there is provided an implantable tube as a structure with a lumen coated with a bioactive material, wherein the bioactive material contains medicament that induces generation of a material promoting fusing of the structure implanted in the body with ambient tissues.

Preferably, the medicament that induces the generation of the material for promoting fusing of the structure, implanted in the body, with ambient tissues is a chemical drug.

In accordance with another exemplary embodiment of the present invention, there is provided a method for coating an implantable tube, including coating a bioactive material on a structure with a lumen. The bioactive material contains a material promoting fusing of the structure implanted in the body with ambient tissues.

Preferably, the material for promoting fusing of the structure, implanted in the body, with ambient tissues is a material for promoting the creation of a blood vessel. The material for promoting fusing of the structure, implanted in the body, with ambient tissues is a material for promoting proliferation of myofibroblast.

Preferably, the bioactive material is coated on an outer surface of the structure. The bioactive material may further include medicament suppressing neointimal hyperplasia.

Preferably, the method may further include coating the medicament suppressing the neointimal hyperplasia on an inner surface of the structure.

Preferably, the material for promoting fusing of the structure, implanted in the body, with ambient tissues is generated by medicament that induces generation of a material promoting fusing with ambient tissues.

Preferably, the medicament that induces the generation of the material for promoting fusing of the structure, implanted in the body, with ambient tissues is a chemical drug.

In accordance with another exemplary embodiment of the present invention, there is provided a method for coating an implantable tube, including coating a bioactive material on a structure with a lumen. The bioactive material contains medicament inducing generation of a material promoting fusing of the structure implanted in the body with ambient tissues.

Preferably, the medicament that induces generation of the material for promoting fusing of the structure, implanted in the body, with ambient tissues is a chemical drug.

### BRIEF DESCRIPTION OF THE DRAWINGS

The features and advantages of the present invention will become more apparent from the following detailed description in conjunction with the accompanying drawings, in which:

FIG. 1 illustrates connecting an artery and vein of a patient for hemodialysis by a tube according to an exemplary embodiment of the present invention;

FIG. 2 illustrates the coating of medicament on the tube according to an exemplary embodiment of the present invention;

FIG. 3 illustrates coated layers formed by coating medicament on the tube according to an exemplary embodiment of the present invention; and

FIG. 4 illustrates the tube with medicament suppressing neointimal hyperplasia coated on an inner surface thereof according to an exemplary embodiment of the present invention.

### BRIEF DESCRIPTION OF SYMBOLS IN THE DRAWINGS

10: tube
11: main body
13: internal space
15-a: a material 15-a for promoting the proliferation of the myofibroblast
15-b: a material 15-b for producing a new blood vessel in the body
17: medicament
A: artery
V: vein

### DETAILED DESCRIPTION OF THE INVENTION

Hereinafter, exemplary embodiments of the present invention are described in detail with reference to the accompanying drawings. The same reference numbers are used throughout the drawings to refer to the same or similar parts. Detailed descriptions of well-known functions and structures incorporated herein may be omitted to avoid obscuring the subject matter of the present invention.

The present invention provides a method of coating a material that promotes fusing a structure with lumen, which has been implanted in the body, with ambient tissues. The embodiment of the present invention is explained based on a case where a structure with lumen is connected with an artery A and vein V of a patient who needs hemodialysis.

A lumen is the inside space of a tubular structure, such as an artery or intestine. In the following description of the present invention, a structure with lumen will be generally referred to as a tube 10.

As shown in FIG. 1, the tube 10 according to an exemplary embodiment of the present invention is implanted in the body to connect an artery A and vein V of the patient who needs hemodialysis. In an embodiment of the present invention, a material that promotes a reaction of fusing the tube 10 and ambient tissues is coated on an outer surface of the tube 10 so that the tube 10, which has been implanted in the body, can be properly fixed to the artery A and vein V. In that case, a material that promotes the creation of a blood vessel and proliferation of myofibroblast or medicament that induces the generation of a growth factor promoting the fusion of the tube 10 with ambient tissues may be coated on the tube 10. This will be described as follows.

In general, when the patient is implanted with the tube 10, he or she is subjected to a stabilization period of two to four weeks until the first dialysis is performed. This period is required to allow the anastomosis part to heal, the implanted tube 10 to be fixed with the ambient tissues, and the blood vessels or nerves to contact with each other through an internal space 13 of the tube 10.

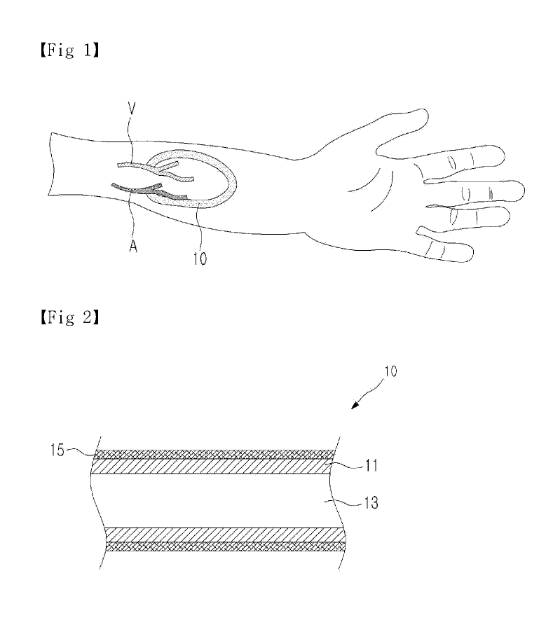

Therefore, as shown in FIG. 2, in order to shorten such a stabilization period, a bioactive material 15 is coated on an outer surface of a main body 11 to promote a reaction of fusing the device with ambient tissues after being implanted in the body. The bioactive material 15 may include a material promoting the creation of blood vessels, a material promoting proliferation of myofibroblast, and medicament inducing the generation of a growth factor promoting fusion with ambient tissues. In this case, the main body 11 is a structure with lumen before the bioactive material 15 is coated thereon.

Meanwhile, the material promoting proliferation of the myofibroblast includes Vascular Endothelial Growth Factor (VEGF), Epidermal Growth Factor (EGF), Platelet Derived Growth Factor (PDGF), Fibroblast Growth Factor (FGF), and the like. In addition, any other material having the same function may also be used.

In this case, VEGF, a vascular endothelial growth factor, increases the transmission of plasma protein in a capillary vessel to promote cell division and movement and induces protease causing cell reconstruction. The VEGF also maintains the existence of a newly formed blood vessel by suppressing cell extinction and promotes the movement of blood vessel cells to promote generation and differentiation of cells.

EGF, a factor promoting the growth of an epithelial cell, induces cell division to promote the growth of the epithelial cell, and plays a key role in restoring skin cells such as promoting cell proliferation of fibroblast synthesizing collagen, a constituent of inner skin, promoting the generation of a new blood vessel in damaged skin, and inducing the secretion of other restoring promotion factors.

PDGF, a blood platelet inducing growth factor, helps divide cells in fibroblast, combines tissues of flexible muscle and also helps recover and reproduce never tissue cells.

FGF, one of heparin-combined growth factors promoting the growth of cells, is used to promote cell proliferation and development in the human body to promote the treatment of wounds. In particular, FGF induces the recovery of large wounds.

Material such as VEGF, EGF, PDGF, and FGF are classified as growth factors, and the medicament inducing generation of such growth factors can be easily coated on the outer surface of the main body 11.

In an embodiment of the present invention, the medicament that induces the generation of the growth factors is a chemical drug, so the medicament can be easily coated on the main body 11 and the tube 10 coated with the chemical drug can be implanted in the body.

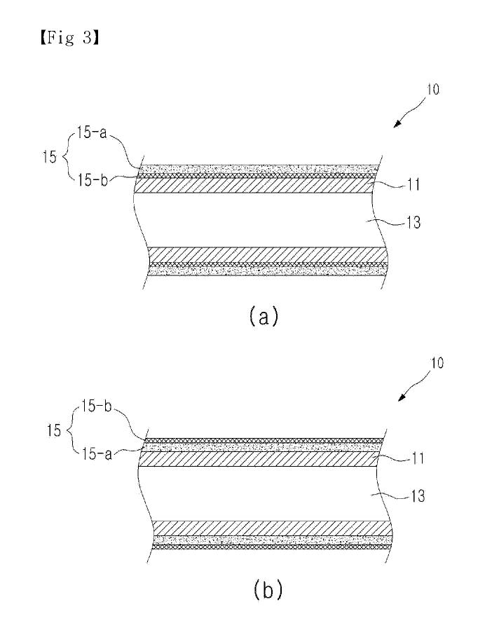

Meanwhile, as shown in FIG. 3, a coated layer 15 is formed as the bioactive material is coated on the outer surface of the main body 11. In that case, as shown in FIG. 3A, a material 15-b for producing a new blood vessel in the body may be first coated and a material 15-a for promoting the proliferation of the myofibroblast between the tube 10 and the ambient tissues may be then coated, or as shown in FIG. 3, the material 15-a for promoting proliferation of the myofibroblast between the main body 11 and the ambient tissues may be first coated, and the material 15-b for newly producing a blood vessel in the body may be then coated.

Alternatively, the material 15-a for promoting the proliferation of the myofibroblast between the main body 11 and the ambient tissues, and the material 15-b for producing a new blood vessel in the body may be respectively coated. Therefore, the tube 10 coated on the main body 11 is generated.

Also, medicament that induces the generation of a growth factor may be coated to promote fusion with the ambient tissues. The medicament may be coated on the outer surface of the main body 11, and may be coated together with the material 15-b for producing a new blood vessel in the body or the material 15-a for promoting the proliferation of the myofibroblast between the main body 11 and the ambient tissues.

The medicament inducing generation of a growth factor may be easily coated on the main body 11 and induct generation of a growth factor promoting fusion with ambient tissues to shorten a stabilization period required after implantation is made.

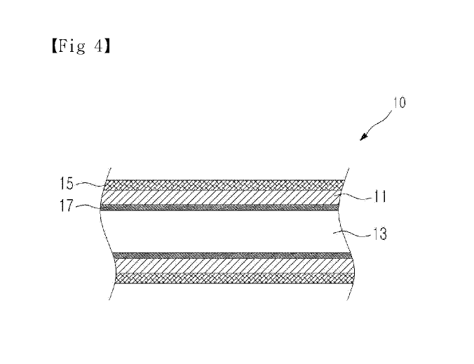

Meanwhile, as shown in FIG. 4, medicament 17 that suppresses neointimal hyperplasia may be coated on the main body 11. The medicament 17 serves to suppress neointimal hyperplasia or thrombosis, so the generation of edema at the site connecting the implanted tube 10 and the ambient tissues or the generation of a state in which a blood vessel is constricted thereby fail to serve as an artificial blood vessel can be prevented.

In this case, the material serving to reduce constriction or the generation of infection may be paclitaxel, rapamycin, taclorimus, cyclosporine A, and the like, and besides, any other material may be used as long as it is medicament that can suppress the constriction of a blood vessel or generation of infection.

It is preferable that the neointimal hyperplasia is coated only on the inner surface of the main body 11. The neointimal hyperplasia may be coated on both outer and inner surfaces of the main body 11.

Meanwhile, in the method for coating medicament suppressing neointimal hyperplasia, a mixed solvent including a material for suppressing neointimal hyperplasia can be used, and in this case, volatile methylene chloride, acetone, etc., may be used.

In addition, in order to generate the tube 10 by coating the main body 11, a bioactive material may be coated on the outer surface of the main body 11 to promote fusion with ambient tissues. If such a material is protein, a coating method using gel or wrap may be used.

On the other hand, in order to facilitate coating, a chemical drug, medicament inducing generation of a growth factor, may be used, and also in this case, a spray method in which a coating solution using an organic solvent may be sprayed on the structure that is used. When only the outer surface of the structure is coated with the coating solution, a dipping method in which the structure is immersed in the coating solution with both ends of the structure covered can be used.

Meanwhile, the tube 10 is formed of a thin film of Expanded Polytetrafluoroethylene (e-PTFE) having fine pores. It is obtained by elongating PTFE in various directions through extrusion at high temperature under high pressure. Also, since e-PTFE has a very low frictional coefficient, it is antithrombotic thereby delaying the adhesion of protein when in contact with blood, so it can be easily used as an artificial blood vessel and also used as an elastic fiber material.

In the exemplary embodiment of the present invention, the tube 10 is described as connecting the artery and vein of the patient who requires hemodialysis, but also in the present invention, the medicament-coated tube 10 may also be used as an artificial lymphatic vessel connecting lymphatic vessels.

For example, the medicament-coated tube 10 may be used to treat diseases such as critical limb ischemia. Alternatively, besides the use as a blood vessel access path, the medicament-coated tube 10 may also be used as a substitute blood vessel such as a coronary artery bypass graft (CAGB).

As described above, according to the present invention, when a structure coated with a bioactive material is implanted in the body, the reaction of fusing and fixing the structure with ambient tissues can be promoted to shorten a stabilization period required until dialysis begins.

Also, since medicament inducing the generation of a material promoting the fusing of the structure with ambient tissues is coated on the structure to generate a material promoting the fusing of the structure with the ambient tissues, when the structure is implanted in the body, the reaction of fusing and fixing the structure with ambient tissues can be promoted to shorten a stabilization period required until dialysis begins.

In addition, medicament inducing generation of the material promoting the fusion of the structure with ambient tissues is a chemical drug, therefore the coating of the medicament inducing the generation of the material promotes the fusing of the structure with ambient tissues.

As the present features may be embodied in several forms without departing from the characteristics thereof, it should also be understood that the above-described embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims.

1. An implantable tube as a structure with a lumen coated with a bioactive material, wherein the bioactive material contains a material promoting fusing of the structure implanted in the body with ambient tissues.

2. The implantable tube of claim 1, wherein the material for promoting fusing of the structure, implanted in the body, with ambient tissues is a material for promoting the creation of a blood vessel.

3. The implantable tube of claim 1, wherein the material for promoting fusing of the structure, implanted in the body, with ambient tissues is a material for promoting the proliferation of myofibroblast.

4. The implantable of claim 3, wherein the material for promoting proliferation of myofibroblast is one of Vascular Endothelial Growth Factor (VEGF), Epidermal Growth Factor (EGF), Platelet Derived Growth Factor (PDGF), and Fibroblast Growth Factor (FGF).

5. The implantable tube of claim 1, wherein the bioactive material is coated on an outer surface of the structure.

6. The implantable tube of claim 1, wherein the bioactive material further comprises medicament suppressing neointimal hyperplasia.

7. The implantable tube of claim 6, wherein the medicament suppressing the neointimal hyperplasia is coated on an inner surface of the structure.

8. The implantable tube of claim 1, wherein the structure is a thin film of Expanded Polytetrafluoroethylene (e-PTFE) having fine pores.

9. The implantable tube of claim 1, wherein the material for promoting fusing of the structure, implanted in the body, with ambient tissues is generated by medicament that induces generation of a material promoting fusing with ambient tissues.

10. An implantable tube as a structure with a lumen coated with a bioactive material, wherein the bioactive material contains medicament that induces generation of a material promoting fusing of the structure implanted in the body with ambient tissues.

11. The implantable tube of claim 9, wherein the medicament that induces the generation of the material for promoting fusing of the structure, implanted in the body, with ambient tissues is a chemical drug.

12. A method for coating an implantable tube, the method comprising:
coating a bioactive material on a structure with a lumen,
wherein the bioactive material contains a material promoting fusing of the structure implanted in the body with ambient tissues.

13. The method of claim 12, wherein the material for promoting fusing of the structure, implanted in the body, with ambient tissues is a material for promoting the creation of a blood vessel.

14. The method of claim 12, wherein the material for promoting fusing of the structure, implanted in the body, with ambient tissues is a material for promoting proliferation of myofibroblast.

15. The method of claim 12, wherein the bioactive material is coated on an outer surface of the structure.

16. The method of claim 12, wherein the bioactive material further comprises medicament suppressing neointimal hyperplasia.

17. The method of claim 16, further comprising:
coating the medicament suppressing the neointimal hyperplasia on an inner surface of the structure.

18. The implantable tube of claim 1, wherein the material for promoting fusing of the structure, implanted in the body, with ambient tissues is generated by medicament that induces generation of a material promoting fusing with ambient tissues.

19.-20. (canceled)

21. The implantable tube of claim 10, wherein the medicament that induces the generation of the material for promoting fusing of the structure, implanted in the body, with ambient tissues is a chemical drug.
